(19) United States
(12) Patent Application Publication
(10) Pub. No.: US 2026/0159289 A1
(43) Pub. Date: Jun. 11, 2026

(54) ATTACHMENT FOR A DRINKING DEVICE FOR RETRONASAL RECEPTION OF AN AROMA SUBSTANCE, AND DRINKING DEVICE

(71) Applicant: air up group GmbH, Munich (DE)

(72) Inventor: Tim Jäger, Munich (DE)

(21) Appl. No.: 18/713,984

(22) PCT Filed: Nov. 30, 2021
(86) PCT No.: PCT/EP2021/083547

§ 371 Date: May 28, 2024

(51) Int. Cl. B65D 51/28; A47G 19/22; A61L 2/10; A61L 9/12; B65D 47/12; B65D 51/24; B65D 81/38

(52) U.S. Cl. CPC B65D 51/28; A47G 19/2272; A47G 19/2288; A61L 2/10; A61L 9/12; B65D 47/123; B65D 51/242; B65D 81/3869; A61L 2202/11; A61L 2209/12; A61L 2209/133; A61L 2209/15

(57) ABSTRACT

An attachment for a drinking device comprises an adapter element to be fluid-tightly attached to a storage container for drinking liquid; a head part to be detachably fastened to the adapter element; an aroma container through which air can flow and which can be fastened to the head part; and a transport channel for drinking liquid, which can be fastened to the head part. The adapter element comprises a first adapter part and a second adapter part. The first adapter part is adapted to be attached to the storage container and to be attached to the second adapter part. The second adapter part comprises a first opening having a first longitudinal axis and a second opening having a second longitudinal axis; wherein the first longitudinal axis is at an angle to the second longitudinal axis, which is at least 20°.

### FIELD OF THE INVENTION

The invention relates to an attachment for a drinking device for the retronasal reception of an aroma substance, and a drinking device with such an attachment.

### PRIOR ART

There is an increasing need to ingest drinking liquids which on the one hand have a pleasant flavor but on the other prevent health risks which could be brought about by the ingestion of aroma substances or stabilizing agents dissolved in the drinking liquid. The ingestion of an increased quantity of calories is also to be avoided.

Water which has been given a slightly fruity aroma has therefore become popular in the last few years. However, in this aromatized water there are also undesired additives such as stabilizing agents and a certain proportion of sugar, which is why these aromatized beverages also have a quantity of calories which is rejected by many users.

A first step towards solving this problem consists in only adding the flavoring aroma to the beverage just before it is consumed. US 2008/028353 A1 and US 2015/030726 A1 are examples of dosing systems with which an aroma substance originally provided separately is added to the drinking liquid and dissolved in it immediately before or during the consumption of the beverage. Although this measure allows problems such as the stabilization of the drinking liquid over a prolonged period of time to be avoided, the problem of the undesired ingestion of additives remains.

Since the olfactory sensation plays a significant part in gustatory perception in the consumption of food and beverages, drinking systems to date have attempted to influence the smell perceived while drinking. To that end U.S. Pat. No. 5,635,229 proposes an aroma element which can be attached close to the drink opening on a drinking container so that the aroma element is in the immediate proximity of the nose of the user, who breathes through the nose while drinking and thereby perceives the aroma. The drinking vessel according to U.S. Pat. No. 8,662,339 B2 also works on the principle that an aroma is inhaled through the nose while drinking.

A drinking device for retronasal ingestion of an aroma substance, comprising an aroma container through which air can flow and which supplies aromatized air to a transport channel for drinking liquid, is known from WO 2019/016096 A1. In this way, the aroma substance is ingested retronasally. During drinking, the aroma substance reaches the mouth of the user together with the drinking liquid and subsequently rises retronasally via the pharynx to the olfactory mucosa, where it is detected by the receptors situated there and is perceived by the user. Advantage is taken of the fact that there is a close correlation between the sense of smell and sense of taste. The user therefore gains the impression that they are tasting the aroma, even though they are in fact only smelling it retronasally.

WO 2020/126210 A1 describes a specific configuration of such a drinking device for the retronasal perception of an aroma substance.

### DESCRIPTION OF THE INVENTION

There is an increasing need to design drinking devices for the retronasal perception of an aroma substance as variably as possible in view of the drinking liquids to be ingested, but also to use these as sustainably as possible.

The invention is therefore based on the object of designing a drinking device for the retronasal perception of an aroma substance more variably with regard to the drinking liquids and more sustainably.

This object is solved by an attachment for a drinking liquid with the features of claim 1 as well as a drinking device for the retronasal perception of an aroma substance with the features of claim 12. Preferred embodiments are apparent from the other claims.

The attachment according to the invention for a drinking device for the retronasal perception of an aroma substance comprises an adapter element to be fluid-tightly attached to a storage container for drinking liquid, a head part to be detachably fastened to the adapter element, and an aroma container through which air can flow and which is fastened to the head part. Moreover, a transport channel for drinking liquid can be fastened to the head part. The adapter element comprises a first adapter part and a second adapter part, wherein the first adapter part is adapted to be attached to the storage container and to be attached to the second adapter part. The second adapter part comprises a first opening having a first longitudinal axis and a second opening having a second longitudinal axis, and the first longitudinal axis is at an angle to the second longitudinal axis, which is at least 20°.

The provision of an adapter element comprising a first adapter part and a second adapter part makes it possible to attach the first adapter part to different storage containers for drinking liquid, since differently shaped first adapter parts can interact with the second adapter part, so that the first adapter parts can each be attached to the second adapter part and can be adapted for different attachment geometries of a storage container for drinking liquid. In the simplest case, these could be different thread shapes or thread diameters that are provided on a storage container for drinking liquid.

Moreover, the adapter element comprising a first adapter part and a second adapter part has the advantage that manufacturing of the adapter element can be simplified since the cranked design of the adapter element with first and second longitudinal axes arranged at an angle with respect to each other is associated with an increased manufacturing effort. Manufacturing the adapter element from two adapter parts also makes it easier to comply with manufacturing tolerances.

Furthermore, in the sense of a modular system, the attachment according to the invention can also be attached to a storage container for drinking liquid, which is made of any material, such as glass, steel or plastic materials. In particular when using a storage container for drinking liquid made of steel, such a container has a longer service life than an attachment made of plastic material, so that the attachment can be replaced. It may also be desirable to replace the attachment according to the invention for hygienic reasons. Since in this event only the attachment, but not the entire drinking device, has to be replaced, the attachment according to the invention contributes to the sustainability of the drinking device provided with the attachment according to the invention.

The first opening of the second adapter part with a first longitudinal axis can serve to detachably fasten the head part, whereas the second opening with a second longitudinal axis represents the side of the attachment, where the first adapter part can be attached. The second longitudinal axis corresponds to the longitudinal axis of a storage container for drinking liquid that can be connected to the attachment.

Arranging the first longitudinal axis at an angle to the second longitudinal axis has the advantage that the head part is arranged at an angle with respect to the storage container for drinking liquid, and thus a drinking device provided with the attachment according to the invention has improved ergonomics and, moreover, it is intuitively clear to a user how to hold the drinking device. Due to the provision of a bend in the extension of the adapter element and thus the drinking device connected with the adapter element, the drinking device is tilted significantly less while drinking, thereby reducing the risk of accidentally spilling the drinking liquid. The provision of an angle between the first longitudinal axis and the second longitudinal axis also makes it possible to replace the drinking container in an upright position without having to tilt the drinking container.

Preferably, the aroma container has an inner cavity enclosed by a container wall, an air inlet opening through the container wall and an air outlet opening through the container wall.

The inner cavity enclosed by the container wall may contain an aroma substance in a suitable manner. For this purpose, a carrier material provided with the aroma substance or a mixture of different aroma substances may be located in the inner cavity of the aroma container. The aroma substance is preferably provided in liquid form.

It is further preferred that, in addition to the carrier material provided with aroma substance, a head space is also located in the inner cavity, which head space is filled with air and serves to enrich the air flowing through the air inlet opening through the container wall as uniformly as possible with aroma substance before it exits through the air outlet opening through the container wall.

The carrier substance in the inner cavity of the aroma container comprises a porous aroma carrier material, which preferably comprises nonwoven fabric. It is further preferred that the air permeability of the porous aroma carrier material at 100 Pa differential pressure is at least 200 l/(m²·s), and preferably between 220 l/(m²·s) and 280 l/(m²·s).

It is further preferred that the porosity of the nonwoven fabric is between 70% and 93%, and preferably between 70% and 80%.

It is further preferred that the Reynolds number Re in the areas of the air inlet opening of the aroma container is at least 2300, wherein the dimensionless Reynolds number is defined as Re=(w·d)/v with the kinematic viscosity of air v[m²/s], the diameter d[m] of the air inlet opening and the flow speed w[m/s] of air when flowing through the air inlet opening into the aroma container at an averages volume flow between 250 ml/min and 600 ml/min.

A Reynolds number of at least 2300 characterizes a turbulent flow or at least a flow which is already in the transition range between a laminar and turbulent flow. A turbulent flow ensures good mixing, as a result of which the uptake of the aroma substance in the air flow flowing through the aroma container is improved. The specified volume flow per minute takes into account the sometimes significant fluctuations in the temporary volume flow when the attachment according to the invention is coupled to a storage container for drinking liquid and a user drinks from the drinking device. Since the drinking process is not a uniform, stationary process, but rather different pressures, but also different flow speeds, prevail in the transport channel for drinking liquid, within the context of the sucking and swallowing processes, there is a significant fluctuation of the temporary volume flows, and thus also flow speeds, at which the air passes through the air inlet opening. Experimentally, however, it has been possible to show that an average volume flow of between 250 ml/min and 600 ml/min flows through an attachment according to the invention with an aroma container, when used as intended. In this case, the physiological drinking process was determined on a large number of different adult people. Since the volume flow is the product of the flow speed and the cross-sectional area, a suitable range for the opening cross-section of the air inlet opening can be determined, under the condition that the Reynolds number describing the flow state must be at least 2300.

According to a preferred embodiment of the invention, the second adapter part is at least sectionally multi-walled, in particular double-walled.

This design has the advantage that the heat insulation of the adapter part is significantly improved and the adapter part is thus also suited for being used with a storage container for drinking liquid, which is filled with drinking liquid that is to remain cold or hot over a prolonged period of time. The double-walled adapter part is particularly suitable for use on a storage container for drinking liquid, which is a thermos vessel.

According to a preferred embodiment of a double-walled adapter part, a UV-light emitting component, including units such as batteries and processors, can be placed in a space between an inner wall and an outer wall of the second adapter part. A UV light-emitting component, for example in the form of a UV diode, can be arranged in such a way that, during operation, it disinfects the space enclosed by the inner wall of the second adapter part through the inner wall of the adapter part. Sensor elements can also be installed and integrated into the adapter, such as a fill level measurement system that can be linked to a smart device via a wireless data connection using NFC. One advantage of integrating functional elements into the adapter, in addition to the modularity of the system, is above all the fast, cost-effective and sustainable replacement of sensor technology, which is subject to rapid technical progress.

According to a preferred embodiment of the invention, the head part has an air channel, the second end of which is in flow connection with an interior space in the transport channel for drinking liquid, and the first end of which is arranged in such a way that the aroma container can be moved into a position in which the air outlet opening of the aroma container is in flow connection with the first end of the air channel.

In this way, the air flowing through the aroma container can enter the air channel directly as it exits the air outlet opening of the aroma container, which opens into the transport channel for drinking liquid. However, the feature according to which the aroma container can be moved into a position in which the air outlet opening is in flow connection with the first end of the air channel also implies in the same way that the aroma container can also be moved into a position in which this flow connection does not exist and thus no air can escape from the aroma container. In this position, no aromatized air enters the transport channel for liquid and the aroma container is in a rest position in which the escape of aromatized air is prevented or at least largely prevented.

According to a preferred embodiment of the invention, the transport channel for drinking liquid has a longitudinal axis which extends in a curved manner.

Since the attachment according to the invention comprises an adapter element, the second adapter part of which is cranked since the first longitudinal axis is at an angle to the second longitudinal axis, and the transport channel for drinking liquid can be fastened to the head part, the cranked extension of the transport channel has the advantage that it can be guided close to the bottom of a storage container for drinking liquid, so that when using the attachment according to the invention on a storage container with drinking liquid having a suitable geometry, the drinking liquid can be sucked up by the user almost entirely.

In this regard, it is advantageous to configure the transport channel for drinking liquid as a drinking straw, the outer cross-section of which is configured in such a way, at least in the area where it is fastened to the head part, that the drinking straw can only be fastened to the head part in a single rotational position in relation to a longitudinal axis. The head part is preferably made of silicone and can hold the inserted drinking straw by friction fit.

The advantage of this design is that, due to the definition of the rotational position during fastening to the head part, the curvature in the extension of the longitudinal axis of the transport channel has a defined orientation which is matched to the overall geometry of the attachment for a drinking device when it is attached to a storage container for drinking liquid.

Preferably, the aroma container is essentially annular with a radially outer boundary wall and a radially inner boundary wall, wherein the radially inner boundary wall encloses a space, the cross-sectional area of which has a geometry deviating from a circular shape.

The provision of an aroma container with an essentially annular geometry has numerous advantages. Firstly, an aroma container of this kind can be fastened around the transport channel for drinking liquid on the head part of the attachment, without the weight of the aroma container being perceived as disruptive when using the attachment on a storage container for drinking liquid since an asymmetrical weight distribution on the drinking device does not occur. Furthermore, providing an annular geometry makes it possible to ensure, by a specific arrangement of the air inlet opening and the air outlet opening, that the air flow flowing through the aroma container travels the longest possible distance and is thus enriched with the aroma substance contained in the aroma chamber as completely as possible.

Since the radially inner boundary wall encloses a space, the cross-sectional area of which has a geometry deviating from a circular shape, correct placement of the aroma container on the attachment can be simplified. If the geometry deviating from a circular shape only allows a single position of the aroma container with respect to its rotation relative to the head part, clear positioning of the aroma container relative to the head part can be ensured. Moreover, the air flowing through the aroma container is mixed better by changes in cross-section and changes of the curvature of the inner cavity of the aroma chamber since all changes in the flow pattern support the occurrence of turbulences.

According to a preferred embodiment of the invention, the adapter element has a receiving means for attaching a carrying strap. The receiving means can be designed as a tongue-and-groove system, for example.

In this way, different carrying straps can be conveniently attached to the adapter element. By choosing the color of the carrying strap, the attachment can be individualized according to the user's wishes, but also when using different first adapter parts for attachment to different storage containers for drinking liquid, a differentiation can be achieved that is easy for a user to grasp.

By arranging the carrying strap at the second adapter part in such a way that the carrying strap is arranged in that area of the second adapter part in which its longitudinal extension is in line with the longitudinal extension of the storage container, the carrying strap can also be used during the drinking process. Moreover, the carrying strap is located at a part of the drinking device that does not rotate when screwing the lid 22 on and off, so that a user can hold the drinking device by the carrying strap while the lid is screwed off or on.

Since the carrying strap is removable, it can be easily replaced in case of damage. Moreover, the carrying strap can serve as an interface to an energy storage device, such as a battery or an accumulator, for example to supply energy to a UV-radiation-emitting unit that requires an associated energy storage device.

Since the second adapter part is shaped in a bended manner, a user can drink comfortably from the mouthpiece without having to tilt the drinking device as a whole. The drinking device can be used in an upright position using the carrying strap, i.e. with the bottom in a horizontal orientation, or the drinking device can be placed on a horizontal surface during the drinking process.

Preferably, the adapter element and the head part are made of plastic. Due to the use of plastic, the adapter element can be easily mass-produced by injection molding. Moreover, a further individualization and color coordination with the carrying strap can be achieved by selecting a suitable color for the plastic material used in addition to the color of the carrying strap. Plastics are particularly suitable since they have a low density and therefore enable lightweight construction, which, in addition to material savings and lower transportation costs, is also perceived as pleasant by users. Plastics also allow greater design freedom due to processing as a melt in the injection molding process. This is not only advantageous for the production of complex geometries in integral design, in which the functionalities of several components are combined in a single component, but also for the reduction of associated production steps.

According to a preferred embodiment, the second adapter part has at least one first positioning element in the area of the first opening, which interacts with a second positioning element provided at the head part, so that the head part can only be attached to the second adapter part in a defined position relative to the first longitudinal axis.

Due to the provision of the at least one first positioning element, the head part can be centered. This is necessary due to the cranked shape of the attachment since a pressure equalizing channel is provided in the head part, which is supposed to be arranged at the upper end of the drinking device during the drinking process so that no drinking liquid can escape from the drinking device. Moreover, a transport channel for drinking liquid, the longitudinal axis of which has a curved extension, can only be used correctly if the head part has been inserted correctly, i.e. the head part has been inserted in the correct orientation. The first positioning element and the second positioning element are preferably one or more ribs and one or more recesses, which also fulfill a guiding function when the head part is attached to the second adapter part, which also makes it easier to attach the head part to the adapter element.

The drinking device according to the invention is preferably made of a different material than the adapter element, preferably the adapter element is made of plastic and the storage container is made of metal.

In this way, the different service lives of the individual modular components can be taken into account or damage to either the storage container for drinking liquid or the attachment can be remedied by replacing only the damaged component, so that the drinking device according to the invention can be used sustainably over a long period of time.

Preferably, the storage container for drinking liquid is at least sectionally double-walled. In this way, the storage container can be used to keep both hot and cold drinking liquids cold or hot for as long as possible. Moreover, a double-shell structure with less material input provides the same mechanical stability as a single-walled storage container. In this way, considerable material savings can be achieved in the mass production of the drinking devices according to the invention.

A suitable positioning and guiding element can also be provided in the area of the connection between the first adapter part and the second adapter part, for example in the form of a guiding rib on the wall of the second adapter part facing the first adapter part, in order to facilitate the attachment of the first adapter part to the second adapter part, in that the first adapter part can only be inserted into the second adapter part in a defined position relative to the latter. Moreover, a form-fitting guiding and positioning means between the first adapter part and the second adapter part can also act in such a way that the second adapter part cannot be rotated relative to the first adapter part, so that a user can automatically detach a first adapter part screwed onto the storage container for drinking liquid from the storage container or screw it onto the storage container by rotating the second adapter part relative to the storage container for drinking liquid.

According to a preferred embodiment of the invention, the head part has an air chamber in connection with the air channel. Said air chamber is a safety buffer for buffering overpressure or negative pressure during and after drinking, and thus it can be avoided that drinking liquid can flow into the aroma container. This solves in particular the problem that the fluctuations in the pressure and flow conditions in the transport channel for drinking liquid and in the air channel in the head part, which occur at the drinking device at the end of the drinking process, can lead to drinking liquid getting into the air channel and/or the aroma container. An ingress of liquid into the aroma container could lead to unwanted dilution of the aroma substance or the occurrence of hygiene problems. The interruption of the air channel by an intermediate air chamber can be configured by providing a recess at the head part of the drinking device where the transport channel for drinking liquid and the air channel come into contact through the head part. If the air chamber extends annularly around the transport channel for drinking liquid, this has the additional advantage that the transport channel for drinking liquid can be fastened to the head part in any rotational position with respect to the longitudinal axis since an air inlet channel into the transport channel for drinking liquid ends in the air chamber and thus the radial orientation does not have to be matched to the air channel in the head part.

The essential aspect of the attachment for a drinking device according to the invention is that the attachment can be placed on different storage containers for drinking liquid and can be adapted independently of the material and the connection geometry of the storage container for drinking liquid by means of a suitable design of the first adapter part.

The storage container for drinking liquid can also be filled with pure water, but also with carbonated water or a drinking liquid having an inherent taste. When using a drinking liquid with inherent taste, the existing inherent taste of the drinking liquid can be either intensified by the aroma substance from the aroma container or supplemented by one or more additional flavor components.

According to a preferred embodiment, the exchangeable aroma container can be moved, in relation to the head part, in an axial direction from a sealing position to a non-sealing position. By means of a simple axial movement of the aroma container, said container can thus be moved from a sealing position to an operating position in which air can flow through the aroma container. When the air inlet opening of the aroma container is closed in the sealing position, the volatilization of the aroma substance into the environment and the ingress of liquid into the aroma container can be prevented.

The attachment according to the invention, comprising the adapter element, can be mounted in the form of a modular system on different storage containers with different connection dimensions and made of different materials. The storage container can be easily filled and refilled since the entire attachment only needs to be detached from the storage container, while the entire attachment with lid and drinking straw can remain in the operating position.

Since experience has shown that a storage container for drinking liquid made of metal, for example, has a longer service life than an attachment made of plastic material, the drinking device can be reused by replacing the attachment and can therefore be used as sustainably as possible.

Due to the shape of the attachment, which can be detached from the storage container for drinking liquid in the area of the first adapter part, the filling opening of the drinking device can be arranged in such a way that it is arranged as closely as possible to the maximum fill level for drinking liquid, making it easier to refill the drinking device, for example under a tap, when there is less space available. In the same way, however, the drinking device can also be filled if there is limited space between the outlet of a tap and a washbasin by removing the head part and refilling water through the first opening in the second adapter part 18 while the longitudinal axis of the storage container is inclined to the vertical.

The drinking device can be used while drinking without having to either hold the drinking device or manually move it into a certain position, making it easy to use even for people with physical limitations.

### BRIEF DESCRIPTION OF THE DRAWINGS

Embodiment examples of the invention are illustrated in the figures. The figures show the following:

FIG. 1 a three-dimensional view of the drinking device;

FIG. 2 a side view of the drinking device illustrated in FIG. 1;

FIG. 3 an exploded view of the drinking device illustrated in FIGS. 1 and 2;

FIG. 4 a sectional view of the drinking device illustrated in FIGS. 1 and 2;

FIG. 5 a sectional view of the two adapter parts of the adapter element;

FIG. 6 a three-dimensional view of the two adapter parts of the adapter element, as shown in FIG. 5;

FIG. 7 the assembled adapter element;

FIG. 8 a sectional view corresponding to the illustration according to FIG. 4 of a further embodiment of the invention; and

FIG. 9 a sectional view through an aroma container.

### DESCRIPTION OF PREFERRED EMBODIMENTS

In the embodiment examples illustrated below, the same components will be designated with the same reference numbers and, where necessary, only the specific differences between the individual embodiments will be discussed.

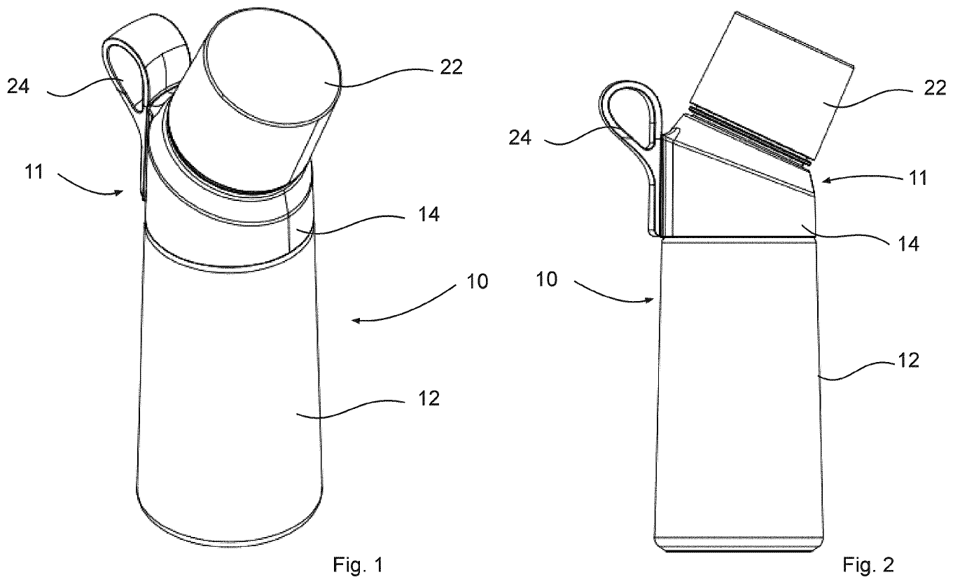

A drinking device according to the invention is illustrated in FIGS. 1 and 2. In this regard, the drinking device 10 comprises a storage container 12 for drinking liquid, an attachment 11 according to the invention as well as a lid 22. A carrying strap 24 is attached to the attachment 11. In the exterior view with the lid screwed on or pushed on, only the adapter part 14 of the attachment 11 is visible. It is apparent that the lid 22 is arranged at an angle with respect to its rotational axis relative to the longitudinal axis of the storage container 12 for drinking liquid, wherein the angle is at least 20°. This arrangement at an angle is created by the geometry of the attachment 11 and in particular the adapter element 14. The adapter element 14 has a bend so that the connecting plane between the adapter element 14 and the storage container 12 is at an angle to the connecting plane between the adapter element 14 and the lid 22.

The adapter element 14 is preferably made of plastic, while the storage container 12 is preferably made of metal and/or glass. However, the storage container 12 can also be made of plastic.

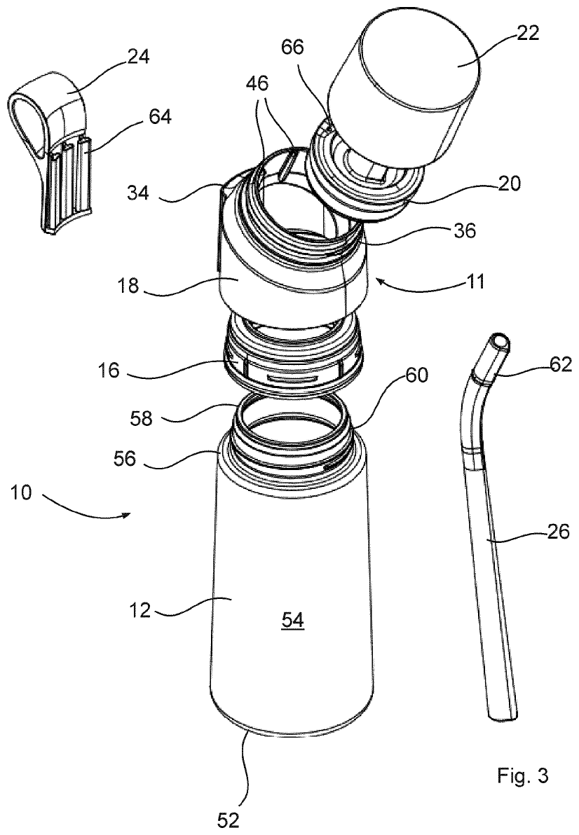

In FIG. 3, the individual elements of the drinking device 10, as illustrated in FIGS. 1 and 2, are shown in an exploded view. The storage container 12 for drinking liquid has an essentially annular bottom 52 as well as a side wall 54 which extends from the bottom 52 upwards. At the upper end of the side wall, an upper surface 56 is located which extends from the side wall 54 essentially horizontally, radially inwards and from which an annular filling opening 58 extends upwards, the outer wall of which is provided with a fastening thread 60. Instead of the fastening thread 60, however, a bayonet-like structure or a suitable latching device could also be provided.

The attachment 11 according to the invention can be screwed onto the fastening thread 60. For this purpose, an adapter element 14, as illustrated in FIGS. 1 and 2, is provided which comprises a first adapter part 16 and a second adapter part 18. The first adapter part 16 is designed in such a way that it can be screwed onto the fastening thread 60 of the storage container 12 and can also be connected to the second adapter part 18. The second adapter part 18 is cranked, i.e. it has a bend, so that the second opening receiving the first adapter part 16 lies in a plane which is arranged at an angle to a first opening which serves to attach a head part 20.

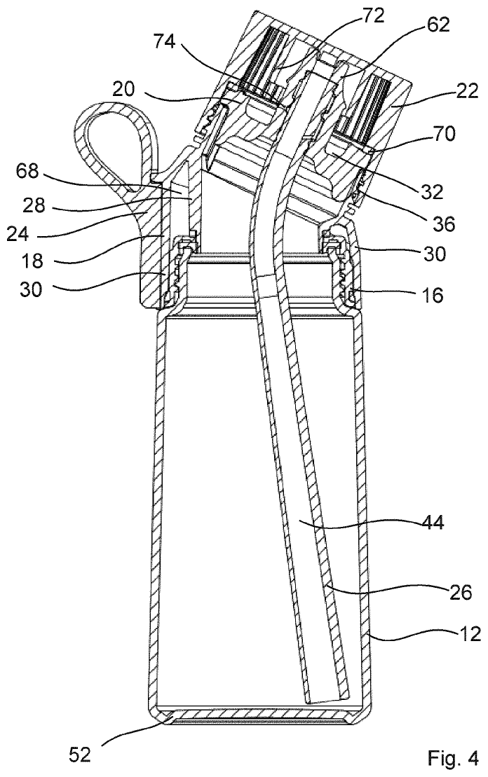

As apparent from the sectional view according to FIG. 4, the head part 20 is provided with a mouthpiece 62 and with a suitable receiving geometry for an aroma container 32. Moreover, the head part 20 is designed in such a way that a drinking straw 26 can be fastened to the head part and can preferably be inserted into the head part.

The drinking straw 26 has a curved extension in the longitudinal direction and moreover has an outer contour at least in the area of the fastening end 62, with which the drinking straw 26 can be fastened to the head part 20, said outer contour allowing, in interaction with an accordingly shaped receiving geometry in the head part, only a single orientation with respect to the rotational axis.

On the second adapter part, a receiving means 34 for a carrying strap is provided, wherein the carrying strap 24 is provided with a complementarily shaped fastening geometry 64 which interacts with the receiving means 34 in such a way that the carrying strap can be pushed in in one direction as far as a stop and is held in this position in a form-fitting manner.

In the area of the first opening 38 of the second adapter part 18, guiding elements 46 are provided which interact with a suitable receiving geometry (not illustrated in FIG. 3) of the head part and thus only allow a single, predefined orientation of the head part 20 relative to the second adapter part 18. A clear orientation of the head part relative to the adapter element is important since the drinking device has a pressure equalizing opening 66 which is supposed to be arranged at the top during the drinking process and serves to let ambient air flow into the drinking device during the drinking process so as to replace the volume of the drinking liquid sucked out of the drinking device.

In the area of the first opening, the second adapter part 18 is provided with an external thread 36 which interacts with an internal thread, not illustrated in FIG. 3, in the lid 22 so as to screw the lid 22 onto the attachment 11.

FIG. 4 shows a sectional view of the drinking device illustrated in an exploded view in FIG. 3. FIG. 4 shows how the aroma container 32 is received in the head part 20 and its annular geometry in a sectional view, however, the geometry of the aroma container 32 deviates from an exact circular ring geometry. Furthermore, it is apparent that the second adapter part 18 is double-walled and comprises an inner wall 28 and an outer wall 30. The double wall of the second adapter part 18 forms a cavity 68 which improves the insulating effect of the second adapter part 18 and thus delays the cooling of hot drinking liquid or the heating of cold drinking liquid. The cavity 68 can also be filled with foam to improve the insulating effect. However, the double wall also serves to reduce the wall thickness while maintaining the same mechanical properties, thus reducing the amount of material required to manufacture the attachment.

The cavity 68 can also be serve to accommodate further elements, such as sensors, batteries, diodes, e.g. for emitting UV light, or heating or cooling elements.

As also apparent from FIG. 4, an annular ledge 70 is formed in the lid, which seals against the head part 20 when the lid 22 is in the screwed-on state. Moreover, a deactivation ring 72 is formed in the lid to move the aroma container 32 downwards relative to the head part 20, when the lid is being closed, and thus putting the aroma container 32 into a resting position in which it is sealed since an air outlet opening from the aroma container 32 is not aligned with an air channel 74 which serves, in the activated position of the aroma container, to convey the aromatized air exiting the aroma container into the transport channel 44 for drinking liquid.

As also illustrated in FIG. 4, the connection in the axial direction between the first adapter part 16 and the second adapter part 18 is a connection by friction fit. However, a snap-in connection could also be established here, which can only be released again, for example, if the locking mechanism is released again by pressing on a specific point on the second adapter element.

As also illustrated in FIG. 4, the drinking straw 26 is inserted into the head part 20 so far that one end of the drinking straw 26 extends to the immediate vicinity of the bottom 52 of the storage container 12 for drinking liquid.

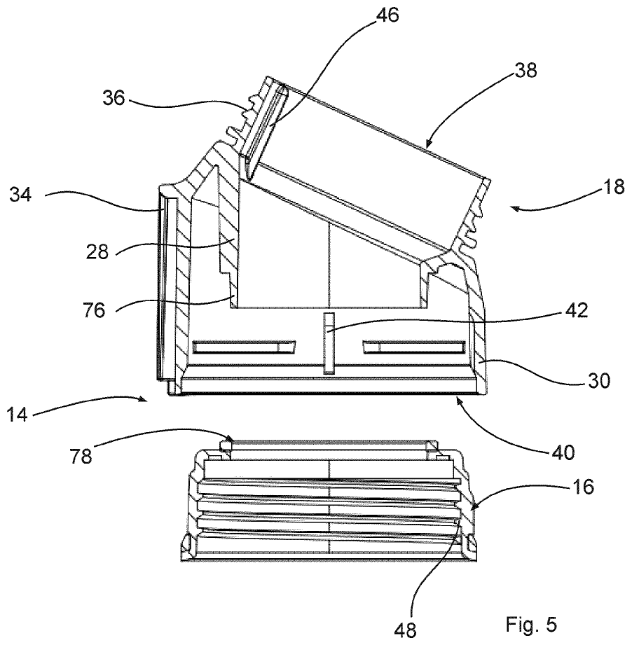

A specific embodiment example of the design of the adapter element 14 comprising the first adapter part 16 and the second adapter part 18 is shown in FIG. 5. In this regard, the inner wall 28 of the second adapter part, the outer wall 30 of the second adapter part, the receiving means 34 for attaching the carrying strap, the at least one guiding element 46 for positioning the head part and the external thread 36 in the area of the first opening 38 of the second adapter part are illustrated in more detail than in the previous embodiments. The angular arrangement of the first opening 38 relative to the second opening 40 is also shown in the same way. The centering and positioning element 42, which prevents unwanted rotation between the first adapter part 16 and the second adapter part 18, can be designed as a simple rib extending in the longitudinal direction, which interacts with an accordingly shaped groove in the first adapter part 16.

The inner wall 28 of the second adapter part 18 is provided with a guide collar 76 which, in the mounted state of the adapter element 14, engages in an upper opening 78 of the first adapter part 16, as also apparent from FIG. 4, and ensures both the stability of the adapter element 14 and the correct positioning of the first adapter part 16 relative to the second adapter part 18.

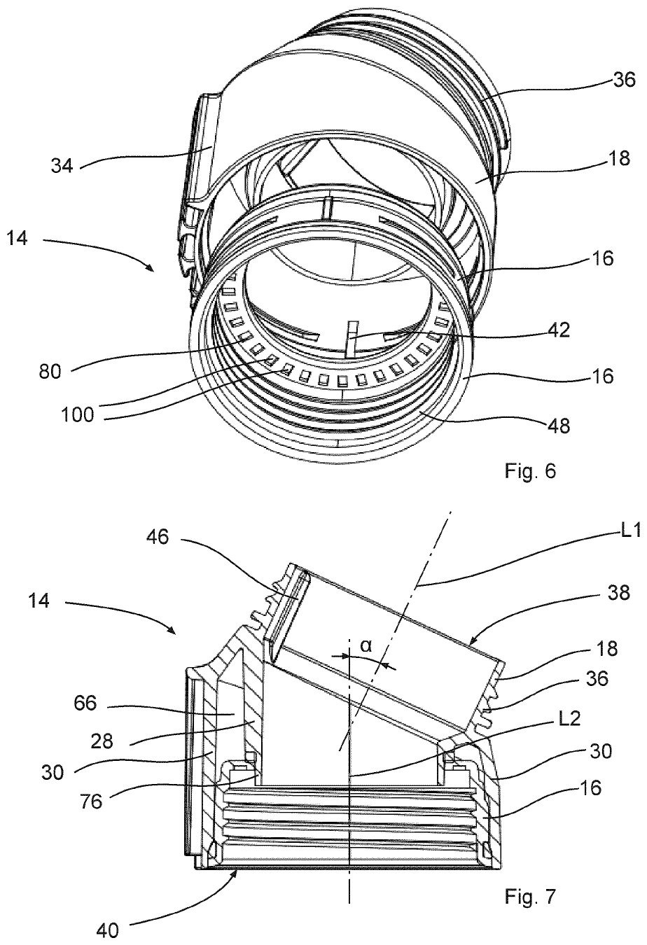

In FIGS. 6 and 7, the adapter element 14 is illustrated again; FIG. 6 is a three-dimensional illustration of FIG. 5, and FIG. 7 shows a sectional view of the adapter element 14 in which the first adapter part 16 is connected with the second adapter part 18, i.e. in the mounted state of the adapter element. In FIG. 7, the angle α of the longitudinal axes L1 and L2 with respect to each other is also illustrated, wherein the first longitudinal axis L1 is perpendicular to the first opening 38 and the second longitudinal axis L2 is perpendicular to the second opening 40, wherein the second longitudinal axis L2 also corresponds to the longitudinal axis of the first adapter part 16. The angle α between the first longitudinal axis L1 and the second longitudinal axis L2 is at least 20° and lies preferably between 20° and 40°.

FIG. 7 moreover shows the interaction of the guide collar 76 of the second adapter part 18 with the upper opening of the first adapter part 16.

FIG. 6 shows a possible shape of the receiving means 34 for attaching the carrying strap, which allows a form-fitting geometry for inserting an accordingly shaped fastening geometry 64 (see FIG. 3) on the carrying strap 24. Moreover, a sealing receptacle 80 is shown in FIG. 6, which serves to accommodate an elastic sealing ring (not shown), which seals in the threaded area between the storage container 12 and the adapter element 14 when the first adapter part 16 is screwed onto the storage container 12.

Distributed around the circumference of the annular sealing receptacle 80, numerous recesses 100 are provided in the sealing receptacle, which are located in the mounted position between the first adapter part 16 and an annular seal inserted into the sealing receptacle 80. The air cushions in the recesses 100 are used to compensate for manufacturing tolerances and improve the sealing effect by compressing the air in the recesses 100 when the adapter element is screwed onto the storage container for drinking liquid. The provision of numerous recesses 100 also increases the service life of the sealing element.

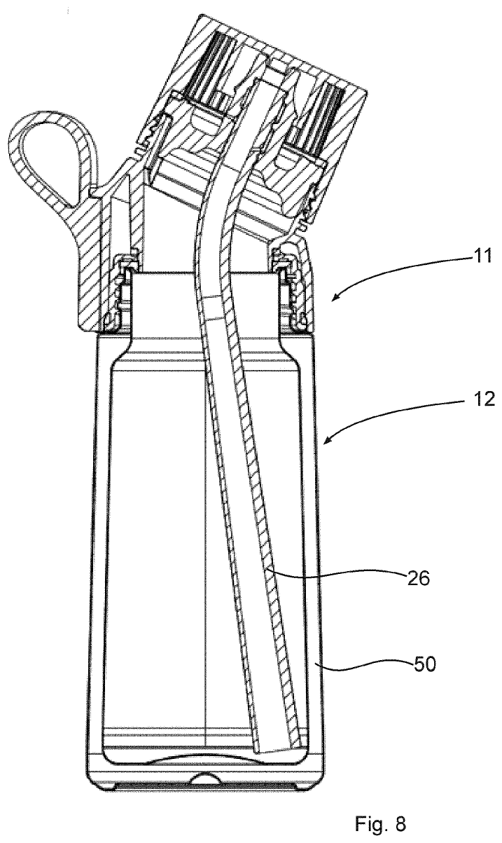

The embodiment according to FIG. 8 largely corresponds to that according to FIG. 4, so that reference can be made to the previous embodiments for an explanation of the attachment 11. However, the difference from the embodiments explained above is that the storage container 12 for drinking liquid is a double-walled vessel 50 designated as insulating vessel or thermos vessel. The double-walled storage container 50 preferably consists of metal, while the attachment 11 is made of plastic. However, other material combinations can also be provided and, for example, the storage container can also be made of glass or a combination of an outer shell made of metal and an inner shell made of mirrored glass.

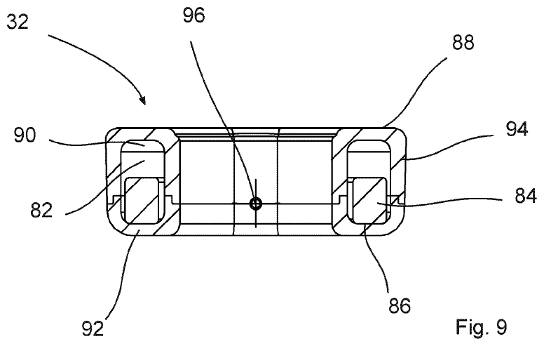

FIG. 9 shows a sectional view of the aroma container 32. The aroma container 32 has an aroma chamber 82 which is formed by an inner cavity 68 in the aroma container 32. In this aroma chamber 82, a carrier substance 84 is located which, in the operating position illustrated in FIG. 8, rests on the lower surface 86 of the aroma chamber 82. Between the carrier substance 84 and the upper surface 88 of the aroma chamber 82, a head space 90 is formed in which the aroma chamber 82 is not filled with the carrier substance 84. Providing the head space 90 has the function of allowing improved enrichment of the air, as well as a homogenization of the air flowing through the aroma container with aroma substance. The head space 90 has a height that is less than 50% of the height of the aroma chamber 82. It is preferred, however, for the carrier substance 84 to occupy at least 80% of the height of the aroma chamber 90.

The aroma container 32 is formed by a lower shell 92 and an upper shell 94. The air inlet opening, which is not shown in the sectional view according to FIG. 9, is located in the lower shell 92, while an air outlet opening 96 is formed in an inner side wall of the essentially annular aroma container. For manufacturing reasons, it has turned out to be advantageous to arrange the air outlet opening 96 at a seam between the lower shell 92 and the upper shell 94, which is advantageous when mass-producing the aroma containers by injection molding.

The carrier substance 84 is a porous aroma carrier material, for example a nonwoven fabric, the porosity of which is preferably between 70% and 93%, and preferably between 70% and 80%. The diameter of the air inlet opening in the lower wall, which is not shown in FIG. 9, is between 1.1 mm and 1.3 mm.

The lower shell 92 and the upper shell 94 can be interconnected by means of ultrasonic welding methods and are connected in a sealed manner circumferentially except in the region of the air outlet opening 96, so that a possible penetration of air into the aroma container as well as an undesired escape of aroma substance from the aroma container 32 can be prevented.

### LIST OF REFERENCE NUMBERS

10 drinking device
11 attachment
12 storage container for drinking liquid
14 adapter element
16 first adapter part
18 second adapter part
20 head part
22 lid
24 carrying strap
26 drinking straw
28 inner wall of the second adapter part
30 outer wall of the second adapter part
32 aroma container
34 receiving means for receiving the carrying strap
36 external thread in the area of the first opening
38 first opening
40 second opening
42 centering and positioning means
44 transport channel for drinking liquid
46 guiding element
48 internal thread of the first adapter part
50 double-walled storage container
52 bottom
54 side wall
56 upper surface
58 filling opening
60 fastening thread
62 fastening end
64 fastening geometry
66 pressure equalizing opening
68 cavity
70 ledge
72 deactivation ring
74 air channel
76 guide collar
78 upper opening
80 sealing receptacle
82 aroma chamber
84 carrier substance
86 lower surface of the aroma chamber
88 upper surface of the aroma chamber
90 head space
92 lower shell
94 upper shell
96 air outlet opening
98 inner side wall
100 recesses

1. An attachment for a drinking device for the retronasal perception of an aroma substance, the attachment comprising:
an adapter configured to be fluid-tightly attached to a storage container for drinking liquid;
a head part configured to be detachably fastened to the adapter;
an aroma container through which air can flow and which is configured to be fastened to the head part; and
a transport channel for drinking liquid, which is configured to be fastened to the head part; wherein
the adapter comprises a first adapter part and a second adapter part; and
the first adapter part is adapted to be attached to the storage container and to be attached to the second adapter part; and
the second adapter part comprises a first opening having a first longitudinal axis and a second opening having a second longitudinal axis; and
the first longitudinal axis is at an angle to the second longitudinal axis, which is at least 20°.

2. The attachment for a drinking device according to claim 1, wherein
the aroma container has an inner cavity enclosed by a container wall, an air inlet opening through the container wall and an air outlet opening through the container wall.

3. The attachment for a drinking device according to claim 2, wherein
the head part has an air channel with first and second ends, the second end of the air channel is in flow connection with an interior space in the transport channel for drinking liquid, and the first end of the air channel is arranged in such a way that the aroma container can be moved into a position in which the air outlet opening is in flow connection with the first end of the air channel.

4. The attachment for a drinking device according to claim 1,
wherein
the second adapter part is at least sectionally multi-walled-walled.

5. The attachment for a drinking device according to claim 4, wherein
a UV-radiation-emitting component can be placed in a cavity between an inner wall and an outer wall of the second adapter part.

6. The attachment for a drinking device according to claim 1,
wherein
the transport channel for drinking liquid has a longitudinal axis which extends in a curved manner.

7. The attachment for a drinking device according to claim 1,
wherein
the transport channel for drinking liquid is a drinking straw, the outer cross-section of the drinking straw is configured in such a way, at least in the area where the drinking straw is fastened to the head part, that the drinking straw can only be fastened to the head part in a single rotational position in relation to a longitudinal axis of the transport channel for drinking liquid.

8. The attachment for a drinking device according to claim 1,
wherein
the aroma container is essentially annular with a radially outer boundary wall and a radially inner boundary wall, wherein the radially inner boundary wall encloses a space, the cross-sectional area of which has a geometry deviating from a circular shape.

9. The attachment for a drinking device according to claim 1,
wherein
the adapter is configured for attachment with a carrying strap.

10. The attachment for a drinking device according to claim 1,
wherein
the adapter and the head part are made of plastic.

11. The attachment for a drinking device according to claim 1,
wherein
in the area of the first opening the second adapter part has at least one first positioning guide interacting with a second positioning guide provided at the head part, so that the head part can only be attached to the second adapter part in a defined rotational position relative to the first longitudinal axis.

12. A drinking device for the retronasal perception of an aroma substance, comprising:
a storage container for drinking liquid; and
an attachment fastened to the storage container for drinking liquid according to claim 1.

13. The drinking device according to claim 12, wherein
the storage container for drinking liquid is made of a different material than the adapter.

14. The drinking device according to claim 12, wherein
the storage container is at least sectionally double-walled.

15. The drinking device according to claim 13, wherein the adapter is made of plastic and the storage container for drinking liquid is made of metal.

16. The attachment for a drinking device according to claim 4, wherein the second adapter part is double-walled.
